(19) United States
(12) Patent Application Publication
(10) Pub. No.: US 2023/0149320 A1
(43) Pub. Date: May 18, 2023

(54) DENSE NANOLIPID FLUID DISPERSIONS COMPRISING TETRAHYDROCANNABINOL AND METHOD OF PREPARATION

(71) Applicant: Eric Morrison, West Saint Paul, MN (US)

(72) Inventor: Eric Morrison, West Saint Paul, MN (US)

(21) Appl. No.: 17/670,445

(22) Filed: Feb. 12, 2022

Related U.S. Application Data
(63) Continuation-in-part of application No. 16/748,399, filed on Jan. 21, 2020, now Pat. No. 11,504,327.
(60) Provisional application No. 63/148,669, filed on Feb. 12, 2021.
(60) Provisional application No. 62/843,763, filed on May 6, 2019.
(60) Provisional application No. 62/794,742, filed on Jan. 21, 2019.

(51) Int. Cl. A61K 9/51; A61K 9/10; A61K 31/352

(52) U.S. Cl. CPC A61K 9/5192; A61K 9/10; A61K 31/352; B82Y 5/00

(57) ABSTRACT

The present invention provides a method of preparing a lipid nanoparticle dispersion using a twin screw extruder. The inventive lipid nanoparticle dispersion includes greater than 25% lipophilic content in the form of lipidic nanoparticles with volume average particle size less than 120 nm dispersed in a continuous aqueous matrix with greater than 0.1 percent of tetrahydrocannabinol.

### RELATED APPLICATIONS

This application claims priority to U.S. Provisional Patent Application Ser. No. 63/148,669 filed Feb. 12, 2021, which claims priority to U.S. Non-Provisional patent application Ser. No. 16/748,399 filed Jan. 21, 2020, which claims priority to U.S. Provisional Patent Application Ser. No. 62/843,763 filed May 6, 2019, 2019, and to U.S. Provisional Patent Application Ser. No. 62/794,742 filed Jan. 21, 2019, all of which are hereby incorporated by reference in their entirety for all purposes.

### BACKGROUND OF THE INVENTION

It is often the case that poorly water-soluble therapeutic agents such as tetrahydrocannabinol are difficult to administer to living organisms in an effective manner because of poor bioavailability.

Tetrahydrocannabinol is hydrogenated 6,6,9-trimethyl-3-pentyl-6H-benzo[c]chromen-1-ol which has potential therapeutic uses including treatment of chronic pain, cancer, nausea and vomiting, loss of appetite, irritable bowel syndrome, epilepsy, Tourette syndrome, Parkinson's disease, Lou Gehrig's disease, Huntington's disease, spasms, dystonia and dyskinesias, dementia, glaucoma, traumatic brain injury, depression, anxiety, posttraumatic stress disorder, and schizophrenia. Because tetrahydrocannabinol can produce euphoric and desirable psychoactive effects, it also finds use as a recreational substance. Tetrahydrocannabinol can take the form of two different structural isomers, delta-8 tetrahydrocannabinol and delta-9 tetrahydrocannabinol each of which has two asymmetric carbons and which may therefore exist in one of four possible stereoisomeric configurations providing a total of eight isomers. As used herein, tetrahydrocannabinol refers to one or more of the eight isomers of hydrogenated 6,6,9-trimethyl-3-pentyl-6H-benzo[c]chromen-1-ol.

The hydrophobic nature of tetrahydrocannabinol for which the calculated value of the logarithm of the octanol/water partition coefficient is equal to 7 contributes to slow physiological absorption and low bioavailability. Although inhalation can give rapid onset starting within minutes and relatively higher bioavailability, disadvantages of inhalation include injury to lung tissue which in severe cases may result in death.

Oral administration, defined as the process by which therapeutic agents are delivered by mouth through the alimentary track, is a safer alternative to inhalation, however it is characterized by slow uptake and low bioavailability. The bioavailability of oral dosage forms of tetrahydrocannabinol is usually less than 20% due to degradation in the stomach, incomplete absorption and excretion, and first-pass hepatic metabolism. The volume of oral dosage forms can be relatively large because of the large human gastric volume which allows using low concentrations of active therapeutic agents, for instance to administer 20 mg of an active therapeutic agent to an individual with a gastric volume of one liter requires a therapeutic agent concentration of 20 ppm. Because low tetrahydrocannabinol concentrations can be useful, it has been practical to use nanotechnology to boost bioavailability by increasing absorption in the alimentary tract and reducing losses due to excretion of unabsorbed tetrahydrocannabinol. Examples of nanotechnology that may be useful for increasing the bioavailability of cannabinoids include example self-emulsifying drug delivery systems (SEDDSs) and “solubilization” of tetrahydrocannabinol by complexation with cyclodextrins.

Unlike hydrophilic water soluble drugs such as ethanol that can be efficiently and rapidly absorbed in the stomach, hydrophobic therapeutic agents such as tetrahydrocannabinol are absorbed in the small intestine. Gastric emptying time typically varies from 0 to 3 hours and the onset time for oral administration of tetrahydrocannabinol may be an hour or more with peak concentrations occurring in 1 to 6 hours. With oral administration of tetrahydrocannabinol, effects commence in a delayed and erratic manner, making it difficult to titrate the required dose resulting in overdosing and underdosing. This often causes individuals taking tetrahydrocannabinol, for example recreational users, to consume more than desired. Even in the case that bioavailability of orally administered tetrahydrocannabinol is improved by providing nano dosage forms, rapidity of onset is not improved. Sublingual and buccal delivery have been suggested as means of administration to improve bioavailability and onset time of therapeutic agents but a constraint of buccal and sublingual dosage forms is they require much higher therapeutic agent concentrations than for oral administration because of the extremely limited volume of oral sublingual and buccal regions, typically about 1 milliliter compared to about 1 liter gastric volume. Administration of 20 mg of a therapeutic agent in a dosage form volume equal to 1 mL requires a therapeutic agent concentration of 2% which is 1000 times greater than is required for oral administration. This has made the use of nanotechnology unfeasible for sublingual and buccal administration of tetrahydrocannabinol, because it has not been possible to provide such high tetrahydrocannabinol concentrations in nanoparticle dispersions.

Unavailability of high tetrahydrocannabinol concentrations in a nanoparticle dispersion form has made it impractical to provide tetrahydrocannabinol cutaneously, i.e. by cutaneous administration, in a nanotechnology enabled form. Cutanous administration of tetrahydrocannabinol is important when it is desirable to keep effects localized near the site of application. The volume of a cutaneously administered product applied to 1000 square centimeters of skin (the approximate area for application to a knee) is between 0.5 and 2.0 mL, and to cutaneously administer 20 mg of a therapeutic agent to an area of 1000 square centimeters requires a tetrahydrocannabinol concentration between 1 and 4%, much higher than can be provided in existing nano dosage forms.

U.S. patent application Ser. No. 16/748,399, METHOD OF PREPARING NANOPARTICLES BY HOT-MELT EXTRUSION which is incorporated in its entirety herein by reference describes a method of making concentrated nanoparticle dispersions that contain between 25% and 65% of lipophilic content where lipophilic content means the sum of the concentrations of surfactants, water immiscible oils, hydrophobic drugs, and hydrophobic bioactive agents. Such highly concentrated dispersions of lipid nanoparticles in water may be termed dense nanolipid fluid (DNLF) dispersions.

Physiologically active compounds such as tetrahydrocannabinol that are included in DNLF dispersions can have high chemical potential, beyond what is possible for microemulsions in which all constituents are at chemical equilibrium. In preferred embodiments, tetrahydrocannabinol DNLF dispersions are not at equilibrium. Evidence that DNLF dispersions are not at equilibrium includes the observation that they do not form spontaneously when all of the constituents are mixed, and the observation that after preparation, they change to a different form such as a DNLF dispersion with a different particle size distribution, for example a particle size distribution with a higher volume average particle diameter, or they phase separate into two or more visibly distinct separate phases. Spontaneous changes, even changes that occur very slowly, demonstrate that DNLF dispersions are not at equilibrium.

### SUMMARY OF THE INVENTION

The present invention generally provides dense nanolipid fluid (DNLF) dispersions comprising tetrahydrocannabinol useful for administration to a mammal by oral, sublingual, buccal or cutaneous administration routes. The DNLF dispersions include: tetrahydrocannabinol, two or more surfactants; one or more water immiscible oils; and water. Methods of preparing DNLF dispersions using a twin screw extrusion method are also provided.

Inventive DNLF dispersions have sufficient concentrations of tetrahydrocannabinol so as to be useful for oral, sublingual, and buccal administration. In preferred embodiments DNLF dispersions have concentrations of tetrahydrocannabinol concentration greater than 0.1%, do not include compounds that are regulated as drugs by the US Food and Drug Administration, and include only edible ingredients. As used herein, compounds that are regulated as drugs by the US Food and Drug Administration do not include cannabinoids such as tetrahydrocannabinol and cannabidiol, even if such compounds may in some cases be regulated as drugs or in the future may become regulated as drugs. In preferred embodiments tetrahydrocannabinol lipid nanoparticle dispersions do not include ibuprofen or S-ibuprofen. In preferred embodiments tetrahydrocannabinol lipid nanoparticle dispersions do not include lidocaine.

In preferred embodiments, tetrahydrocannabinol lipid nanoparticle dispersions are not at equilibrium and have high kinetic stability such that they do not phase separate within 28 days when stored at temperatures between 18° C. and 23° C. and they do not phase separate within 7 days when stored at 40° C. In preferred embodiments, lipid nanoparticle dispersions do not form spontaneously within 2 weeks after oil phase and aqueous phase after low shear mixing of an oil phase with an aqueous phase. Low shear mixing includes mixing using a magnetic stir bar and magnetic stirrer and stirring using a stirring element including a blade, a paddle, or a spiral paint mixer such as a Red Devil® 5 Gallon Drywall Mud/Paint Mixer Model Number 4041 or equivalent.

Preferred lipid nanoparticle dispersions have latent lamellar structure, that is, the propensity for nanoparticle dispersions without lamellar structure to develop lamellar structure. Latent lamellar structure can be evaluated by inducing changes in nanoparticle dispersions such as by evaporation or heating and observing for characteristics of lamellar structures such as optical birefringence and electrical impedance. Optical birefringence can be observed by viewing through crossed polarized films. Electrical impedance can be observed as a negative peak in a plot of conductivity versus temperature and a corresponding positive peak in the first derivative plot of conductivity vs temperature for heated and stirred nanoparticle dispersions when heating at a rate of between 1 and 4° C. per minute and measuring conductivity with an open cell geometry electrode such as a 013005MD 4-cell conductivity electrode available from Thermo Scientific.

Suitable high hydrophile-lipophile-balance (HLB) surfactants useful in the practice of the current invention include ester type polyethoxylated high HLB surfactants such as polysorbate-80, polysorbate-60, polysorbate-20, PEG100 stearate and PEG-30 glyceryl cocoate; ether type polyethoxylated high HLB surfactants such as steareth-40, laureth-23 and ceteareth-30; anionic high HLB surfactants comprising sulfate and carboxylate groups such as sodium lauryl sulfate, sodium stearoyl lactylate, and salts of fatty acids such as sodium octanoate. Suitable low hydrophile-lipophile-balance (HLB) surfactants useful in the practice of the current invention include phospholipid compounds such as lecithins and phosphatidyl choline compounds such as dioleylphosphatidyl choline; esters of sorbitan with fatty acids such as sorbitan stearate and sorbitan oleate; esters of glycerin with fatty acids such as glyceryl monostearate and mono- and di-glycerides; and fatty acids such as stearic acid.

Suitable water immiscible oils useful in the practice of the current invention include triglyceride oils such as medium chain triglyceride oil, ester oils such as methyl undecylenate, ethyl caproate, ethyl caprylate, ethyl myristate, ethyl oleate and isopropyl myristate; aliphatic hydrocarbons such as light mineral oil, terpenes such as d-limonene, myrcene, eugenol, humulene, and olive oil unsaponifiables; and botanical extracts and essential oils such as arnica flower oil, orange essential oil, clove essential oil, and cinnamon bark essential oil.

Lipid nanoparticle dispersions of the present invention comprise one or more polyethoxylated high hydrophile-lipophile-balance (HLB) surfactants such as polysorbate-80, polysorbate-60, polysorbate-20, PEG-30 glyceryl cocoate, steareth-40, ceteareth-30, laureth-23 and PEG100 stearate; one or more low hydrophile-lipophile-balance (HLB) surfactants such as lecithin, glyceryl esters such as glyceryl monostearate, sorbitan esters such as sorbitan stearate and sorbitan oleate, fatty acids such as octanoic acid and stearic acid, mono- and di-glycerides, and fatty alcohols such as lauryl alcohol and stearoyl alcohol; one or more water immiscible oils such as hydrocarbons, ester oils such as medium chain triglyceride oil and isopropyl myristate, terpenes such as d-limonene and humulene, and essential oils such as arnica flower oil and clove essential oil; and water. Lipid nanoparticle dispersions of the present invention may optionally include non polyethoxylated high hydrophile-lipophile-balance (HLB) surfactants such as sodium lauryl sulfate, sodium stearoyl lactylate, and sodium octanoate.

The present invention provides a lipid nanoparticle dispersion suitable for oral, sublingual, buccal or cutaneous administration to a mammal. The nanoparticle dispersion includes greater than 0.1 percent tetrahydrocannabinol; from about 10 to about 16 percent of one or more polyethoxylated high hydrophile-lipophile-balance (HLB) surfactants; from about 4 to about 8 percent of one or more low hydrophile-lipophile-balance (HLB) surfactants; from about 25 to about 40 percent of one or more water immiscible oils; and from about 30 to about 60 percent water.

In one embodiment, the lipid nanoparticle dispersion has greater than 45 percent lipophilic content.

In one embodiment, the lipid nanoparticle dispersion further comprises one or more non polyethoxylated high hydrophile-lipophile-balance (HLB) surfactants.

In one embodiment, the lipid nanoparticle dispersion further comprises one or more non polyethoxylated high hydrophile-lipophile-balance (HLB) surfactants selected from the group of sodium lauryl sulfate, sodium stearoyl lactylate, and sodium octanoate.

In one embodiment, the lipid nanoparticle dispersion comprises lipid nanoparticles that are not vesicular nanoparticles.

In one embodiment, the lipid nanoparticle dispersion comprises lipid nanoparticles that do not have a lamellar structure.

In one embodiment, the lipid nanoparticle dispersion has a latent lamellar structure.

In one embodiment, the lipid nanoparticle dispersion further comprises cannabidiol.

In one embodiment, the lipid nanoparticle dispersion further comprises one or more oil soluble vitamins.

In one embodiment, the lipid nanoparticle dispersion comprises one or more oil soluble vitamins selected from the group of ascorbyl palmitate and tocopherol.

In one embodiment, the lipid nanoparticle dispersion further includes one or more cryoprotectants.

In one embodiment, the lipid nanoparticle dispersion comprises one or more cryoprotectants selected from the group of glycerin and propylene glycol.

In one embodiment, the lipid nanoparticle dispersion further comprises one or more preservatives.

In one embodiment, the lipid nanoparticle dispersion comprises one or more preservatives selected from the group of butylated hydroxy toluene, sodium benzoate, and sodium phytate.

In one embodiment, the lipid nanoparticle dispersion is free of ether type polyethoxylated high hydrophile-lipophile-balance (HLB) surfactants.

In one embodiment, the lipid nanoparticle dispersion is free from ibuprofen, S-ibuprofen and lidocaine.

In one embodiment, the weight ratio of ester type polyethoxylated high HLB surfactants to ether type polyethoxylated high hydrophile-lipophile-balance (HLB) surfactants is less than 1 to 1.

In one embodiment, the turbidity of the lipid nanoparticle dispersion is less than 750 NTU.

In one embodiment, the turbidity of the lipid nanoparticle dispersion is less than 500 NTU.

In one embodiment, the turbidity of the lipid nanoparticle dispersion is less than 200 NTU.

In one embodiment, the lipid nanoparticle dispersion has volume average particle size less than 75 nm.

In one embodiment, the lipid nanoparticle dispersion is stable to phase separation when stored for 7 days at 40° C.

In one embodiment, tetrahydrocannabinol comprises (6aR,10aR)-6,6,9-trimethyl-3-pentyl-6a,7,8,10a-tetrahydrobenzo[c]chromen-1-ol.

In one embodiment, tetrahydrocannabinol comprises (6aR,10aR)-6,6,9-trimethyl-3-pentyl-6a,7,10,10a-tetrahydrobenzo[c]chromen-1-ol.

In one embodiment, the concentration of tetrahydrocannabinol is greater than 1%.

In one embodiment, the sum of the concentration of cannabinoid compounds is greater than 2.5%.

The present invention provides a lipid nanoparticle dispersion for oral, sublingual, buccal or cutaneous administration to a mammal. The nanoparticle dispersion includes greater than 0.1 percent tetrahydrocannabinol; one or more high hydrophile-lipophile-balance (HLB) surfactants selected from the group consisting of polysorbate-80, polysorbate-60, polysorbate-20, sodium lauryl sulfate, sodium stearoyl lactylate, PEG-30 glyceryl cocoate, steareth-40, ceteareth-30, laureth-23, PEG100 stearate, and sodium octanoate; one or more low hydrophile-lipophile-balance (HLB) surfactants selected from the group consisting of lecithin, glyceryl monostearate, sorbitan stearate, sorbitan oleate, stearic acid, and mono- and di-glycerides; one or more water immiscible oils selected from the group consisting of medium chain triglyceride oil, methyl undecylenate, ethyl caproate, ethyl caprylate, ethyl myristate, ethyl oleate, isopropyl myristate, light mineral oil, d-limonene, myrcene, eugenol, humulene, olive oil unsaponifiables, arnica flower oil, orange essential oil, clove essential oil, and cinnamon bark essential oil; water, wherein said lipid nanoparticle dispersion has a volume average particle size less than about 120 nm and greater than 25 percent lipophilic content.

In one embodiment, the lipid nanoparticle dispersion has greater than 45 percent lipophilic content.

In one embodiment, the lipid nanoparticle dispersion comprises lipid nanoparticles that are not vesicular nanoparticles.

In one embodiment, the lipid nanoparticle dispersion comprises lipid nanoparticles that do not have a lamellar structure.

In one embodiment, the lipid nanoparticle dispersion has a latent lamellar structure.

In one embodiment, the lipid nanoparticle dispersion further comprises cannabidiol.

In one embodiment, the lipid nanoparticle dispersion further comprises one or more oil soluble vitamins.

In one embodiment, the lipid nanoparticle dispersion comprises one or more oil soluble vitamins selected from the group of ascorbyl palmitate and tocopherol.

In one embodiment, the lipid nanoparticle dispersion further includes one or more cryoprotectants.

In one embodiment, the lipid nanoparticle dispersion comprises one or more cryoprotectants selected from the group of glycerin and propylene glycol.

In one embodiment, the lipid nanoparticle dispersion further comprises one or more preservatives.

In one embodiment, the lipid nanoparticle dispersion comprises one or more preservatives. selected from the group of butylated hydroxy toluene, sodium benzoate, and sodium phytate.

In one embodiment, the lipid nanoparticle dispersion is free of ether type polyethoxylated high hydrophile-lipophile-balance (HLB) surfactants.

In one embodiment, the lipid nanoparticle dispersion is free from ibuprofen, S-ibuprofen and lidocaine.

In one embodiment, the weight ratio of ester type polyethoxylated high HLB surfactants to ether type polyethoxylated high hydrophile-lipophile-balance (HLB) surfactants is less than 1 to 1.

In one embodiment, the turbidity of the lipid nanoparticle dispersion is less than 750 NTU.

In one embodiment, the turbidity of the lipid nanoparticle dispersion is less than 500 NTU.

In one embodiment, the turbidity of the lipid nanoparticle dispersion is less than 200 NTU.

In one embodiment, the lipid nanoparticle dispersion has volume average particle size less than 75 nm.

In one embodiment, the lipid nanoparticle dispersion is stable to phase separation when stored for 7 days at 40° C.

In one embodiment, tetrahydrocannabinol comprises (6aR,10aR)-6,6,9-trimethyl-3-pentyl-6a,7,8,10a-tetrahydrobenzo[c]chromen-1-ol.

In one embodiment, tetrahydrocannabinol comprises (6aR,10aR)-6,6,9-trimethyl-3-pentyl-6a,7,10,10a-tetrahydrobenzo[c]chromen-1-ol.

In one embodiment, the concentration of tetrahydrocannabinol is greater than 1%.

In one embodiment, the sum of the concentration of cannabinoid compounds is greater than 2.5%.

The present invention provides a lipid nanoparticle dispersion for oral, sublingual, or buccal administration to a mammal comprising; greater than 0.1 percent tetrahydrocannabinol; one or more high hydrophile-lipophile-balance (HLB) surfactants selected from the group consisting of polysorbate-80, polysorbate-60, polysorbate-20, sodium lauryl sulfate, sodium stearoyl lactylate, PEG-30 glyceryl cocoate, and sodium octanoate; one or more low hydrophile-lipophile-balance (HLB) surfactants selected from the group consisting of lecithin, glyceryl monostearate, sorbitan stearate, sorbitan oleate, stearic acid, and mono- and di-glycerides; one or more water immiscible oils selected from the group consisting of medium chain triglyceride oil, methyl undecylenate, ethyl caproate, ethyl caprylate, ethyl myristate, ethyl oleate, isopropyl myristate, light mineral oil, d-limonene, myrcene, eugenol, humulene, olive oil unsaponifiables, arnica flower oil, orange essential oil, clove essential oil, and cinnamon bark essential oil; water, wherein the lipid nanoparticle dispersion has a volume average particle size less than about 120 nm and greater than 25 percent lipophilic content.

In one embodiment, the lipid nanoparticle dispersion has greater than 45 percent lipophilic content.

In one embodiment, the lipid nanoparticle dispersion comprises lipid nanoparticles that are not vesicular nanoparticles.

In one embodiment, the lipid nanoparticle dispersion comprises lipid nanoparticles that do not have a lamellar structure.

In one embodiment, the lipid nanoparticle dispersion has a latent lamellar structure.

In one embodiment, the lipid nanoparticle dispersion further comprises cannabidiol.

In one embodiment, the lipid nanoparticle dispersion further comprises one or more oil soluble vitamins.

In one embodiment, the dispersion comprises one or more oil soluble vitamins includes selected from the group of ascorbyl palmitate and tocopherol.

In one embodiment, the lipid nanoparticle dispersion further comprises glycerin.

In one embodiment, the lipid nanoparticle dispersion further comprises one or more preservatives.

In one embodiment, the lipid nanoparticle dispersion further comprises one or more preservatives selected from the group of butylated hydroxy toluene and sodium benzoate.

In one embodiment, the lipid nanoparticle dispersion is free from ether type polyethoxylated high hydrophile-lipophile-balance (HLB) surfactants.

In one embodiment, the lipid nanoparticle dispersion is free from ibuprofen, S-ibuprofen and lidocaine.

In one embodiment, all surfactants, water immiscible oils, essential oils, oil soluble vitamins, cryo-protectants, and preservatives present in the lipid nanoparticle dispersion amounts greater than 0.1% are listed in the US Food and Drug Administration Code of Federal Regulations Title 21 Part 172 Food additives permitted for direct addition to food for human consumption.

In one embodiment, the lipid nanoparticle dispersion has turbidity less than 750 NTU.

In one embodiment, the turbidity of the lipid nanoparticle dispersion is less than 500 NTU.

In one embodiment, the turbidity of the lipid nanoparticle dispersion is less than 200 NTU.

In one embodiment, the lipid nanoparticle dispersion has volume average particle size less than 75 nm.

In one embodiment, the lipid nanoparticle dispersion is stable to phase separation when stored for 7 days at 40° C.

In one embodiment, tetrahydrocannabinol comprises (6aR,10aR)-6,6,9-trimethyl-3-pentyl-6a,7,8,10a-tetrahydrobenzo[c]chromen-1-ol.

In one embodiment, tetrahydrocannabinol comprises (6aR,10aR)-6,6,9-trimethyl-3-pentyl-6a,7,10,10a-tetrahydrobenzo[c]chromen-1-ol.

In one embodiment, the concentration of tetrahydrocannabinol is greater than 1%.

In one embodiment, the sum of the concentration of cannabinoid compounds is greater than 2.5%.

The present invention provides a lipid nanoparticle dispersion for cutaneous administration to a mammal comprising greater than 0.1 percent tetrahydrocannabinol; one or more high hydrophile-lipophile-balance (HLB) surfactants selected from the group consisting of polysorbate-80, ceteareth-30, laureth-23, PEG100 stearate, and steareth-40; one or more low hydrophile-lipophile-balance (HLB) surfactants selected from the group consisting of lecithin, glyceryl monostearate, sorbitan oleate, and stearic acid; one or more water immiscible oils selected from the group consisting of medium chain triglyceride oil, isopropyl myristate, light mineral oil, and arnica flower oil; water, wherein the lipid nanoparticle dispersion has a volume average particle size less than about 120 nm and greater than 25 percent lipophilic content.

In one embodiment, the lipid nanoparticle dispersion has greater than 45 percent lipophilic content.

In one embodiment, the lipid nanoparticle dispersion comprises lipid nanoparticles that are not vesicular nanoparticles.

In one embodiment, the lipid nanoparticle dispersion comprises lipid nanoparticles that do not have a lamellar structure.

In one embodiment, the lipid nanoparticle dispersion has a latent lamellar structure.

In one embodiment, the lipid nanoparticle dispersion further comprises cannabidiol.

In one embodiment, the lipid nanoparticle dispersion further comprises one or more oil soluble vitamins.

In one embodiment, the lipid nanoparticle dispersion further comprises one or more oil soluble vitamins selected from the group of ascorbyl palmitate and tocopherol.

In one embodiment, the lipid nanoparticle dispersion further comprises one or more cryoprotectants.

In one embodiment, the lipid nanoparticle dispersion further comprises one or more cryoprotectants selected from the group of glycerin and propylene glycol.

In one embodiment, the lipid nanoparticle dispersion further comprises one or more preservatives.

In one embodiment, the lipid nanoparticle dispersion further comprises one or more preservatives selected from the group of butylated hydroxy toluene, sodium benzoate, and sodium phytate.

In one embodiment, the weight ratio of ester type polyethoxylated high hydrophile-lipophile-balance (HLB) surfactants to ether type polyethoxylated high hydrophile-lipophile-balance (HLB) surfactants is in the lipid nanoparticle dispersion is less than 1 to 1.

In one embodiment, the lipid nanoparticle dispersion has turbidity less than 750 NTU.

In one embodiment, the turbidity of the lipid nanoparticle dispersion is less than 500 NTU.

In one embodiment, the turbidity of the lipid nanoparticle dispersion is less than 200 NTU.

In one embodiment, the lipid nanoparticle dispersion has volume average particle size less than 75 nm.

In one embodiment, the lipid nanoparticle dispersion is stable to phase separation when stored for 7 days at 40° C.

In one embodiment, tetrahydrocannabinol comprises (6aR,10aR)-6,6,9-trimethyl-3-pentyl-6a,7,8,10a-tetrahydrobenzo[c]chromen-1-ol.

In one embodiment, tetrahydrocannabinol comprises (6aR,10aR)-6,6,9-trimethyl-3-pentyl-6a,7,10,10a-tetrahydrobenzo[c]chromen-1-ol.

In one embodiment, the concentration of tetrahydrocannabinol is greater than 1%.

In one embodiment, the sum of the concentration of cannabinoid compounds is greater than 2.5%.

The present invention provides a process for the preparation of a lipid nanoparticle dispersion wherein said process comprises conveying a mill base through a twin screw extruder with a process ΔT of greater than about 45° C. wherein said mill base comprises greater than 0.1 percent tetrahydrocannabinol; from about 10 to about 16 percent of one or more polyethoxylated high hydrophile-lipophile-balance (HLB) surfactants; from about 4 to about 8 percent of one or more low hydrophile-lipophile-balance (HLB) surfactants; from about 25 to about 40 percent of one or more water immiscible oil; and from about 30 to about 60 percent water and wherein the lipid nanoparticle dispersion has a volume average particle size less than 120 nm.

In one embodiment, the lipid nanoparticle dispersion has a latent lamellar structure.

In one embodiment, the twin screw extruder barrel comprises an internal cooling channel.

In one embodiment, a fluid with temperature less than 10° C. is pumped through the internal cooling channel of the twin screw extruder barrel during processing of a mill base.

In one embodiment, the extruder outlet temperature is less than 25° C.

In one embodiment, the process temperature change is greater than 65° C.

In one embodiment, the mill base forms a lamellar structure microemulsion between 60° C. and 100° C.

In one embodiment, the mill base is free from ether type polyethoxylated high hydrophile-lipophile-balance (HLB) surfactants.

In one embodiment all surfactants, water immiscible oils, essential oils, oil soluble vitamins, cryo-protectants, and preservatives present in the mill base with concentrations greater than 0.1% are listed in the US Food and Drug Administration Code of Federal Regulations Title 21 Part 172 Food additives permitted for direct addition to food for human consumption.

In one embodiment, the mill base is free from ibuprofen, S-ibuprofen and lidocaine.

In one embodiment, tetrahydrocannabinol comprises (6aR,10aR)-6,6,9-trimethyl-3-pentyl-6a,7,8,10a-tetrahydrobenzo[c]chromen-1-ol.

In one embodiment, tetrahydrocannabinol comprises (6aR,10aR)-6,6,9-trimethyl-3-pentyl-6a,7,10,10a-tetrahydrobenzo[c]chromen-1-ol.

In one embodiment, the concentration of tetrahydrocannabinol is greater than 1%.

In one embodiment, the sum of the concentration of cannabinoid compounds is greater than 2.5%.

The present invention provides a process for the preparation of a lipid nanoparticle dispersion wherein said process comprises conveying a mill base through a twin screw extruder with a process ΔT of greater than about 45° C. wherein said mill base comprises greater than 0.1 percent tetrahydrocannabinol; one or more high hydrophile-lipophile-balance (HLB) surfactants selected from the group consisting of polysorbate 80, polysorbate 60, polysorbate-20, sodium lauryl sulfate, sodium stearoyl lactylate, PEG-30 glyceryl cocoate, steareth-40, ceteareth-30, laureth-23, PEG100 stearate, and sodium octanoate; one or more low hydrophile-lipophile-balance (HLB) surfactants selected from the group consisting of lecithin, glyceryl monostearate, sorbitan stearate, sorbitan oleate, stearic acid, and mono- and di-glycerides; one or more water immiscible oils selected from the group consisting of medium chain triglyceride oil, methyl undecylenate, ethyl caproate, ethyl caprylate, ethyl myristate, ethyl oleate, isopropyl myristate, light mineral oil, d-limonene, myrcene, eugenol, humulene, olive oil unsaponifiables, arnica flower oil, orange essential oil, clove essential oil, and cinnamon bark essential oil; and water and wherein the lipid nanoparticle dispersion has a volume average particle size less than 120 nm.

In one embodiment, the twin screw extruder barrel comprises an internal cooling channel.

In one embodiment, a fluid with temperature less than 10° C. is pumped through the internal cooling channel of the twin screw extruder barrel during processing of a mill base.

In one embodiment, the extruder outlet temperature is less than 25° C.

In one embodiment, the Δtemperature change is greater than 65° C.

In one embodiment, the mill base forms a lamellar structure microemulsion between 60° C. and 100° C.

In one embodiment, the mill base is free from ether type polyethoxylated high hydrophile-lipophile-balance (HLB) surfactants.

In one embodiment all surfactants, water immiscible oils, essential oils, oil soluble vitamins, cryo-protectants, and preservatives present in the mill base with concentrations greater than 0.1% are listed in the US Food and Drug Administration Code of Federal Regulations Title 21 Part 172 Food additives permitted for direct addition to food for human consumption.

In one embodiment, the mill base is free from ibuprofen, S-ibuprofen and lidocaine.

In one embodiment, tetrahydrocannabinol comprises (6aR,10aR)-6,6,9-trimethyl-3-pentyl-6a,7,8,10a-tetrahydrobenzo[c]chromen-1-ol.

In one embodiment, tetrahydrocannabinol comprises (6aR,10aR)-6,6,9-trimethyl-3-pentyl-6a,7,10,10a-tetrahydrobenzo[c]chromen-1-ol.

In one embodiment, the concentration of tetrahydrocannabinol is greater than 1%.

In one embodiment, the sum of the concentration of cannabinoid compounds is greater than 2.5%.

### DETAILED DESCRIPTION OF THE INVENTION

As used herein, the term “tetrahydrocannabinol” refers to a single isomer of hydrogenated 6,6,9-trimethyl-3-pentyl-6H-benzo[c]chromen-1-ol or a mixture of isomers of hydrogenated 6,6,9-trimethyl-3-pentyl-6H-benzo[c]chromen-1-ol.

As used herein, “delta-9 tetrahydrocannabinol” refers to the compound with IUPAC name (6aR,10aR)-6,6,9-trimethyl-3-pentyl-6a,7,8,10a-tetrahydrobenzo[c]chromen-1-ol.

As used herein, “delta-8 tetrahydrocannabinol” refers to the compound with IUPAC name (6aR,10aR)-6,6,9-trimethyl-3-pentyl-6a,7,10,10a-tetrahydrobenzo[c]chromen-1-ol.

As used herein, “cannabinoid” and “cannabinoid compound” refers to a naturally occurring compounds found in the Cannabis sativa plant that interact with the cannabinoid receptors of the body and brain.

As used herein, the term “lipid” refers to fats and fat-derived materials which are insoluble in water except as micellar solutions or dispersions but which are soluble in organic solvents.

As used herein, the term “dense nanolipid fluid (DNLF) dispersion” refers to a lipid nanoparticle dispersion in water with between 25% and 65% of lipophilic content where lipophilic content means the sum of the concentrations of surfactants, water immiscible oils, hydrophobic drugs, and hydrophobic therapeutic agents.

As used herein, “lipophilic content” means the sum of the concentrations of surfactants, water immiscible oils, hydrophobic drugs, and hydrophobic bioactive agents.

As used herein, the term “dispersion of lipid nanoparticles in water” refers to a dispersion of lipid particles in water with a volume average particle size less than 150 nm.

As used herein, the term “therapeutic agent” refers to a chemical compound, complex or composition that exhibits a desirable effect in the biological context, i.e., when administered to a subject.

As used herein, the term “drug” refers to a chemical compound that is regulated as a drug by the US Food and Drug Administration except for cannabinoids such as tetrahydrocannabinol and cannabidiol, even if such compounds may in some cases be regulated as drugs or in the future may become regulated as drugs.

As used herein, the term “oral administration” refers to the process by which drugs are delivered by mouth through the alimentary track.

As used herein, the term “buccal administration” refers to the process by which therapeutic agents are held or applied in the buccal area and diffuse through the oral mucosa directly into the bloodstream.

As used herein, the term “sublingual administration” refers to the process by which therapeutic agents are held or applied to the area under the tongue and diffuse through the oral mucosa directly into the bloodstream.

As used herein, the term “cutaneous administration” refers to the process by which therapeutic agents are applied to the skin.

As used herein, the term “HLB” refers to Hydrophile-Lipophile-Balance, which is an empirical expression for the relationship of the hydrophilic (“water-loving”) and hydrophobic (“water-hating”) groups of a surfactant.

As used herein, the phrase “low hydrophile-lipophile-balance (HLB) surfactant” refers to a surfactant with an hydrophile-lipophile-balance (HLB) value of less than about 10.

As used herein, the phrase “ether type polyethoxylated high hydrophile-lipophile-balance (HLB) surfactant” refers to a surfactant with an hydrophile-lipophile-balance (HLB) value of equal to or greater than about 14 and an ether linkage between hydrophilic and hydrophobic groups in the surfactant.

As used herein, the phrase “ester type polyethoxylated high hydrophile-lipophile-balance (HLB) surfactant” refers to a surfactant with an hydrophile-lipophile-balance (HLB) value of equal to or greater than about 14 and an ester linkage between hydrophilic and hydrophobic groups in the surfactant.

As used herein, the phrase “anionic high hydrophile-lipophile-balance (HLB) surfactant” refers to a surfactant with a hydrophile-lipophile-balance (HLB) value of equal to or greater than about 10 that dissociates in water to give an anion comprising a hydrophobic group covalently bonded to an anionic group such as a sulfate or carboxylate group plus a cation selected from the group of hydrogen ion and alkali metal ions.

As used herein, “cryoprotectant” refers to compounds that are used to retard crystalline ice formation in compositions that contain water upon cooling.

As used herein, “preservative” refers to a substance or a chemical that is added to products such as food products, beverages, and drugs to prevent decomposition by microbial growth or by undesirable chemical changes.

As used herein, the term “essential oil” refers to a volatile oil derived from the leaves, stem, flower or twigs of plants or synthetically-made compounds that have the same chemical attributes. The essential oil usually carries the odor or flavor of the plant.

As use herein, the term “immiscible” refers to liquids that will not mix or remain mixed with each other, although at certain conditions, for example, high temperatures, they might mix, but any such mixture will typically be thermodynamically unstable and will typically separate into distinct phases at lower temperatures.

As used herein, “lamellar structure” refers to a structure resulting from packing of amphipathic molecules in an environment of a polar liquid as bilayers with polar groups of the amphipathic molecules in contact with the polar liquid.

As used herein, “latent lamellar structure” refers to lamellar structure that is not observable in a dispersion including surfactants, oil and water but becomes observable when the dispersion undergoes heating or evaporation.

As used herein, “process ΔT” refers to the extruder inlet temperature minus the extruder outlet temperature.

As used herein, “extruder inlet temperature” refers to the maximum of the temperature of a mill base as it enters the extruder barrel and the maximum temperature of the extruder temperature control zones.

As used herein, “extruder outlet temperature” refers to the temperature of a mill base as it exits the extruder barrel.

As used herein, “mill base” refers to a composition comprising one or more polyethoxylated high hydrophile-lipophile-balance (HLB) surfactants, one or more low hydrophile-lipophile-balance (HLB) surfactants, one or more water immiscible oils, and water with between about 25 and about 65 percent lipophilic content.

### EXAMPLES

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

### Sources of Materials

| Description of Material | Sources of Material |
|---|---|
| polysorbate 80 | Modernist Pantry LLC, |
| | Eliot, Maine |
| polysorbate 60 | Lotioncrafter, |
| | Eastsound WA |
| polysorbate 20 | Florida Laboratories, |
| | Fort Lauderdale, FL |
| sodium stearoyl lactylate | Modernist Pantry LLC, |
| | Eliot, Maine |
| sodium lauryl sulfate | Nature's Oil, |
| | Aurora, OH |
| octanoic acid | Proctor and Gamble, |
| | Cincinnati, OH |
| ceteareth-30 | Jeen International Corporation, |
| | Fairfield, NJ |
| laureth-23 | Lotioncrafter, |
| | Eastsound WA |
| steareth-40 | Ethox Chemicals, |
| | Greenville, SC |
| lecithin (Alcolec XTRA-A | American Lecithin, |
| soy lecithin) | Oxford, CT |
| glyceryl monostearate | Modernist Pantry LLC, |
| | Eliot, Maine |
| mono- and di-glycerides | Modernist Pantry LLC, |
| | Eliot, Maine |
| sorbitan stearate | Lotioncrafter, |
| | Eastsound WA |
| sorbitan oleate | Jeen International Corporation, |
| | Fairfield, NJ |
| delta-8 tetrahydrocannabinol | Fresh Brothers Hemp Co., |
| | Las Vegas, NV |
| | Primordia LLC, |
| | Brawley, CA |
| cannabidiol | Mile High Labs, |
| | Broomfield, CO |
| 93.6% delta-9 | Curative Health |
| tetrahydrocannabinol | Cultivation LLC, |
| (Indica Blend) | Aurora IL |
| 88.8% delta(9)- | Ingrown Farms LLC, |
| tetrahydrocannabinolic acid | Ridott, IL |
| (Aurora Chem Haze) | |
| medium chain triglyceride oil | Physician's Choice, |
| | Westminster, CO |
| light mineral oil (Drakeol 7) | CQ Concepts, |
| | Ringwood, IL |
| isopropyl myristate | Jeen International Corporation, |
| | Fairfield, NJ |
| | Soaper's Choice, |
| | Des Plaines, IL |
| ethyl caproate | Vigon International Inc., |
| | East Stroudsburg, PA |
| ethyl caprylate | Vigon International Inc., |
| | East Stroudsburg, PA |
| ethyl myristate | Vigon International Inc., |
| | East Stroudsburg, PA |
| myrcene | Vigon International Inc., |
| | East Stroudsburg, PA |
| humulene | Vigon International Inc., |
| | East Stroudsburg, PA |
| d-limonene | Florida Laboratories, |
| | Fort Lauderdale, FL |
| olive oil unsaponifiables | Lotioncrafter, |
| | Eastsound WA |
| arnica flower oil | Salvia Nutrition, |
| | Vendée, FR |
| eugenol | Vigon International Inc., |
| | East Stroudsburg, PA |
| clove essential oil | Now Foods, |
| | Bloomingdale, IL |
| cinnamon bark essential oil | Pranarōm USA, |
| | Golden Valley, MN |
| concentrated orange essential oil | The Essential Oil Company, |
| | Portland, OR |
| rosemary - mint fragrance | Elements Bath & Body Supply, |
| | Pueblo, CO |
| ascorbyl palmitate | Solaray, |
| | Park City, UT |
| tocopherol (Vitapherole 50) | The Herbarie, |
| | Prosperity, SC |
| BHT | Loud Wolf, |
| | Dublin CA |
| citric acid | Lotioncrafter, |
| | Eastsound WA |
| hydrochloric acid | Home Science Tools, |
| | Billings, MT |
| sodium phytate | Lotioncrafter, |
| | Eastsound WA |
| glycerin | Lotioncrafter, |
| | Eastsound WA |
| propylene glycol | Lotioncrafter, |
| | Eastsound WA |
| sodium benzoate | Lotioncrafter, |
| | Eastsound WA |
| Jeecide CAP-4 preservative | Jeen International Corporation, |
| | Fairfield, NJ |
| niacinamide | PureBulk, |
| | Roseburg OR |
| stearic acid | Soaper's Choice, |
| | Des Plaines, IL |
| PEG100 stearate (Myrj S100) | Croda, |
| | Princeton NJ |
| dioleylphosphatidyl choline | Lipoid, |
| (Phospholipon 90G) | Newark NJ |

Mill bases for processing with a modified twin screw extruder were prepared by warming oil phase ingredients including tetrahydrocannabinol, surfactants, water immiscible oils, and hydrophobic bioactive agents to about 60° C. and adding aqueous phase ingredients including water and water soluble ingredients such as salts, citric acid, glycerin and propylene glycol while stirring using a Red Devil® 5 Gallon Drywall Mud/Paint Mixer Model Number 4041 spiral paint mixer with a hand held electric drill.

A Leistritz 27 mm twin screw extruder was modified by removing the cooling water manifold and the hoses between the manifold and the barrel. Fittings screwed into the barrel connecting the barrel to cooling water manifold hoses were replaced with hose barbs and ½ inch ID braided flexible clear PVC tubing was used to connect the motor end entrance of each barrel section cooling channel to the die end entrance of the cooling channel of the preceding (closer to the motor) section of the extruder barrel. Propylene glycol/water coolant was circulated through the extruder barrel in reverse order (that is, zone 9 then zone 8, zone 7, zone 6, zone 5, zone 4 and zone 3) using a Lauda T10000 chiller with set point=0.1° C. The barrel zones set point temperatures were set to 10° C. and no heat was provided to the barrel from the extruder controller. The screw configuration (in units of length over diameter starting at the motor end of the extruder was): L/D 0.0 to L/D 1.1=15 mm pitch intermeshing conveying, L/D 1.1 to L/D 12.2=40 mm pitch intermeshing conveying, L/D 12.2 to L/D 24.4=30 mm pitch intermeshing conveying, and L/D 24.4 to L/D 36.7=40 mm pitch intermeshing conveying. Before processing each Example, the extruder was prepared by processing a composition consisting of 8.2% polysorbate 80, 5.5% polysorbate 20, 2.3% soy lecithin, 4.1% glyceryl monostearate, 1.4% stearic acid, 13.6% isopropyl myristate, 9.1% medium chain triglyceride oil, 9.1% light mineral oil, 0.1% butylated hydroxy toluene, 0.9% Jeecide CAP-4 preservative, 0.2% sodium benzoate, 0.04% citric acid, and 45.5% water that had been heated to 90° C. and admitted to entrance of the extruder barrel at zone 1 using a screw rotation rate of 150 rpm or 200 rpm until each temperature zone in the extruder barrel reached a constant temperature. Prior to admitting to the extruder barrel entrance at zone 1, the mill base for each Example was heated to 92±7° C. The temperature of the lipid nanoparticle dispersion product of each example was less than 25° C. at the exit of the extruder barrel and the delta T for processing each example (defined as the temperature of the mill base upon entering the extruder at zone 1 minus the extrudate temperature was greater than 60° C.

The particle size distributions of the extruded lipid nanoparticle dispersions were measured within two days of preparation on samples diluted approximately 50 times with deionized water using a NanoFlex dynamic light scattering (DLS) instrument (Microtrac Instruments, York Pa.).

Particle polydispersity was estimated as the ratio of volume average particle size (Dv) to number average particle size (Dn).

Lipid nanoparticle dispersion samples were tested for latent lamellar structure by heating while measuring conductivity and observing for birefringence. Approximately 40 g sample of lipid nanoparticle dispersion in a 50 mL beaker with a magnetic stirrer is carefully warmed using a 900 watt microwave oven on 40% setting to between 45 and 50° C. to reduce the viscosity and allow the sample to stir on a hotplate stirrer. The sample is then stirred using a hotplate stirrer while heating at a rate of 2 to 3° C. per minute while logging the conductivity with a Thermo Scientific Orion 3-star Conductivity Meter Model 1114000 with a 013005MD 4-cell conductivity cell electrode and observing through two crossed pieces of polarizing film. Latent lamellar structure is evident as a negative peak in the plot of conductivity vs temperature and/or birefringence which can be observed as a pattern of alternating darker and lighter regions or as colored regions in a stirring sample that is transparent, isotropic and featureless when observed without polarizing film.

Turbidity of lipid nanoparticle dispersions was measured within two days of extrusion by placing extruded fluids into 1 cm×1 cm, 3.5 mL polystyrene cuvettes polished on four sides and turbidity measured using a Neulog NUL-231 turbidity sensor and USB-200 interface (products of SES Education, Rishon Lezion, Israel) and averaging readings on all four possible cuvette orientations. If the standard deviation of readings was greater than 5% of the average reading, samples in cuvettes are centrifuged at 2900 relative centrifugal force for 10 minutes which is effective to remove air bubbles. Turbidity was calculated from the instrument reading using a standard curve prepared from 4000 NTU formazin turbidity standard (catalog number 246142, product of Hach Company, London Ontario) freshly diluted to 300 nephelometric turbidity units (NTU).

### Example 1

### Processing of an Edible Mill Base Including Polysorbate 80, Glyceryl Monostearate, Sorbitan Oleate, Isopropyl Myristate, Light Mineral Oil, Tetrahydrocannabinol, Ascorbyl Palmitate, Tocopherol, Glycerin, and Water Using a Modified Twin Screw Extruder to Give an Edible DNLF Dispersion

A mill base was prepared by warming a mixture of oil phase components consisting of 15.6% polysorbate 80, 1.1% glyceryl monostearate, 2.0% sorbitan oleate, 27.1% isopropyl myristate, 10.9% light mineral oil, 1.0% delta 8 tetrahydrocannabinol, 0.3% ascorbyl palmitate, 0.7% tocopherol and 0.1% clove essential oil to about 60° C. to give a hazy transparent liquid, adding a clear aqueous phase solution with 0.2% sodium benzoate, 0.0% citric acid, 1.1% glycerin, and 40.0% water, and mixing with a Red Devil® 5 Gallon Drywall Mud/Paint Mixer Model Number 4041 spiral paint mixer using a hand held electric drill. The ratio of ether type polyethoxylated high HLB surfactant to ester type polyethoxylated high HLB surfactant was 0:100. The mill base was processed with an extruder inlet temperature equal to 91° C., extruder outlet temperature equal to 18° C. and process ΔT=73° C. at a rate of 500 grams per minute to give a tetrahydrocannabinol lipid nanoparticle dispersion as a translucent soft psuedoplastic gel. The volume average particle diameter (Dv) was 70 nm, the number average particle diameter (Dn) was 40 nm and the polydispersity was 1.8. When a sample of the lipid nanoparticle dispersion was tested for latent lamellar structure, the plot of conductivity vs temperature shows a negative peak with maximum depression of conductivity at 74° C. and the sample was birefringent between 57 and 69° C. and again between 75 and 79° C. which indicates that it has latent lamellar structure. When stored at 40° C., the sample was stable to phase separation for at least 23 days.

This example demonstrates that processing a composition comprising tetrahydrocannabinol, oil soluble vitamins, a single ester type polyethoxylated high HLB surfactant, a single low HLB surfactant, and a single ester oil is effective to produce an edible tetrahydrocannabinol containing DNLF dispersion with latent lamellar structure and volume average particle diameter less than 100 nm.

### Example 2

### Processing of an Edible Mill Base Including Polysorbate 80, Polysorbate 20, Sodium Stearoyl Lactylate, Soy Lecithin, Isopropyl Myristate, Medium Chain Triglyceride Oil, Light Mineral Oil, Tetrahydrocannabinol, and Water Using a Modified Twin Screw Extruder

A mill base was prepared consisting of 41.8% water, 15.0% isopropyl myristate, 9.6% light mineral oil, 9.6% medium chain triglyceride oil, 8.7% polysorbate 80, 5.7% polysorbate 20, 4.2% sodium stearoyl lactylate, 2.4% soy lecithin, 1.8% delta 8 tetrahydrocannabinol, 0.3% hydrochloric acid 0.3% clove essential oil, 0.2% sodium benzoate, 0.1% butylated hydroxy toluene, and 0.04% citric acid. The ratio of ether type polyethoxylated high HLB surfactant to ester type polyethoxylated high HLB surfactant was 0:100. The mill base was processed with an extruder inlet temperature equal to 90° C., extruder outlet temperature equal to 22° C. and process ΔT=68° C. at a rate of 470 grams per minute to give a tetrahydrocannabinol lipid nanoparticle dispersion as a translucent soft psuedoplastic gel. The volume average particle diameter (Dv) was 76 nm, the number average particle diameter (Dn) was 45 nm, and the polydispersity was 1.7. When stored at 40° C., the sample was stable to phase separation for at least 23 days.

This example demonstrates that processing a composition comprising tetrahydrocannabinol, a mixture of ester type polyethoxylated high HLB surfactants, a single low HLB surfactant, a single aliphatic hydrocarbon oil and a single ester oil is effective to produce an edible tetrahydrocannabinol containing lipid nanoparticle dispersion with volume average particle diameter less than 100 nm.

### Example 3

### Processing of an Edible Mill Base Including Polysorbate 80, Polysorbate 20, Sodium Stearoyl Lactylate, Soy Lecithin, Ethyl Oleate, Medium Chain Triglyceride Oil, Light Mineral Oil, Cannabidiol, Tetrahydrocannabinol and Water Using a Modified Twin Screw Extruder

A mill base was prepared consisting of 40.8% water, 14.5% ethyl oleate, 9.7% light mineral oil, 9.7% medium chain triglyceride oil, 8.7% polysorbate 80, 5.8% polysorbate 20, 4.2% sodium stearoyl lactylate, 2.4% soy lecithin, 1.8% delta 8 tetrahydrocannabinol, 1.5% cannabidiol, 0.3% clove essential oil, 0.2% sodium benzoate, 0.1% hydrochloric acid 0.1% butylated hydroxy toluene, and 0.04% citric acid. The ratio of ether type polyethoxylated high HLB surfactant to ester type polyethoxylated high HLB surfactant was 0:100. The mill base was processed with an extruder inlet temperature equal to 88° C., extruder outlet temperature equal to 23° C. and process ΔT=65° C. at a rate of 510 grams per minute to give a tetrahydrocannabinol lipid nanoparticle dispersion as a translucent soft psuedoplastic gel. The volume average particle diameter (Dv) was 74 nm, the number average particle diameter (Dn) was 55 nm and the polydispersity was 1.3. When a sample of the lipid nanoparticle dispersion was tested for latent lamellar structure, the plot of conductivity vs temperature did not show a negative peak and the sample was birefringent between 58 and 67° C. which indicates that it has latent lamellar structure. When stored at 40° C., the sample phase separated sometime between 20 and 23 days.

This example demonstrates that processing a composition comprising tetrahydrocannabinol, a blend of ester type polyethoxylated high HLB surfactants, a single low HLB surfactant, and a single ester oil (ethyl oleate) is effective to produce an edible tetrahydrocannabinol containing DNLF dispersion with latent lamellar structure and volume average particle diameter less than 100 nm.

### Example 4

### Processing of an Edible Mill Base Including Polysorbate 80, Polysorbate 20, Sodium Stearoyl Lactylate, Soy Lecithin, Medium Chain Triglyceride Oil, Light Mineral Oil, Ethyl Caproate, Isopropyl Myristate, Ethyl Caprylate, Tetrahydrocannabinol and Water Using a Modified Twin Screw Extruder

A mill base was prepared consisting of 41.4% water, 9.8% medium chain triglyceride oil, 9.8% light mineral oil, 8.8% polysorbate 80, 5.9% polysorbate 20, 5.0% ethyl caproate, 4.9% isopropyl myristate, 4.9% ethyl caprate, 4.3% sodium stearoyl lactylate, 2.5% soy lecithin, 1.7% delta 8 tetrahydrocannibinol, 0.3% hydrochloric acid, 0.3% clove essential oil, 0.2% sodium benzoate, 0.1% butylated hydroxy toluene, and 0.04% citric acid. The ratio of ether type polyethoxylated high HLB surfactant to ester type polyethoxylated high HLB surfactant was 0:100. The mill base was processed with an extruder inlet temperature equal to 92° C., extruder outlet temperature equal to 23° C. and process ΔT=69° C. at a rate of 510 grams per minute to give a tetrahydrocannabinol lipid nanoparticle dispersion as a translucent soft psuedoplastic gel. The volume average particle diameter (Dv) was 71 nm, the number average particle diameter (Dn) was 54 nm and the polydispersity was 1.3. When a sample of the lipid nanoparticle dispersion was tested for latent lamellar structure, the plot of conductivity vs temperature shows a negative peak with maximum depression of conductivity at 60° C. and the sample was birefringent between 54 and 63° C. which indicates that it has latent lamellar structure. When stored at 40° C., the sample phase separated sometime between 14 and 16 days.

This example demonstrates that processing a composition comprising tetrahydrocannabinol, a blend of ester type polyethoxylated high HLB surfactants, a single low HLB surfactant, and a mixture of ester oils including ethyl caproate, ethyl caprylate and isopropyl myristate is effective to produce an edible tetrahydrocannabinol DNLF dispersion with volume average particle diameter less than 100 nm.

### Example 5

### Processing of an Edible Mill Base Including Polysorbate 80, Polysorbate 20, Sodium Stearoyl Lactylate, Soy Lecithin, Medium Chain Triglyceride Oil, Light Mineral Oil, Methyl Undecylenate, Isopropyl Myristate, Tetrahydrocannibinol and Water Using a Modified Twin Screw Extruder

A mill base was prepared consisting of 41.3% water, 9.8% light mineral oil, 9.8% medium chain triglyceride oil, 9.8% isopropyl myristate, 8.9% polysorbate 80, 5.9% polysorbate 20, 5.0% methyl undecylenate, 4.2% sodium stearoyl lactylate, 2.5% soy lecithin, 1.8% delta 8 tetrahydrocannibinol, 0.3% hydrochloric acid, 0.3% clove essential oil, 0.2% sodium benzoate, 0.1% butylated hydroxy toluene, and 0.04% citric acid. The ratio of ether type polyethoxylated high HLB surfactant to ester type polyethoxylated high HLB surfactant was 0:100. The mill base was processed with an extruder inlet temperature equal to 92° C., extruder outlet temperature equal to 23° C. and process ΔT=69° C. at a rate of 580 grams per minute to give a tetrahydrocannabinol lipid nanoparticle dispersion as a translucent soft pseudoplastic gel. The volume average particle diameter (Dv) was 65 nm, the number average particle diameter (Dn) was 50 nm and the polydispersity was 1.3. When a sample of the lipid nanoparticle dispersion was tested for latent lamellar structure, the plot of conductivity vs temperature shows a negative peak with maximum depression of conductivity at 61° C. and the sample was birefringent between 54 and 59° C. and again between 62 and 66° C. which indicates that it has latent lamellar structure. The turbidity of the lipid nanoparticle dispersion was 660 NTU. When stored at 40° C., the sample was stable to phase separation for at least 23 days.

This example demonstrates that processing a composition comprising tetrahydrocannabinol, a blend of ester type polyethoxylated high HLB surfactants, a single low HLB surfactant, and a mixture of ester oils including methyl undecylenate is effective to produce an edible tetrahydrocannabinol DNLF dispersion with latent lamellar structure, volume average particle diameter less than 100 nm and turbidity less than 750 NTU.

### Example 6

### Processing of an Edible Mill Base Including Polysorbate 80, Polysorbate 60, Polysorbate 20, Sodium Stearoyl Lactylate, Soy Lecithin, Isopropyl Myristate, d-Limonene, Olive Oil Terpenes, Cannabidiol, Tetrahydrocannibinol and Water Using a Modified Twin Screw Extruder

A mill base was prepared consisting of 36.9% water, 25.0% isopropyl myristate, 6.9% polysorbate 80, 5.0% d-limonene, 4.9% olive oil unsaponifiables, 4.8% polysorbate 60, 3.0% sodium stearoyl lactylate, 2.1% polysorbate 20, 2.0% propylene glycol, 2.0% glycerin, 1.8% delta 8 tetrahydrocannibinol, 1.5% cannabidiol, 1.5% mono- and di-glycerides, 1.2% soy lecithin, 0.5% ascorbyl palmitate, 0.4% clove essential oil, 0.2% sodium benzoate, 0.1% butylated hydroxy toluene, and 0.1% citric acid. The ratio of ether type polyethoxylated high HLB surfactant to ester type polyethoxylated high HLB surfactant was 0:100. The mill base was processed with an extruder inlet temperature equal to 95° C., extruder outlet temperature equal to 19° C. and process ΔT=76° C. at a rate of 580 grams per minute to give a tetrahydrocannabinol lipid nanoparticle dispersion as a transparent soft psuedoplastic gel. The volume average particle diameter (Dv) was 48 nm, the number average particle diameter (Dn) was 40 nm, the polydispersity was 1.2 and the turbidity was 460 NTU. When stored at 40° C., the sample phase separated sometime between 12 and 14 days. A 400 mg sample of the extruded product was applied to the buccal and sublingual oral cavities using an airless pump bottle with a 2 mm OD×1.2 mm ID acrylonitrile-butadiene-styrene applicator tube. Slower absorption resulting from swallowing 200 mg of the extruded sample in a hydroxypropyl methylcellulose capsule (product of Capsule Supplies LLC, Philmont N.Y.) is effective to prolong drug effects and reduce the level of intoxication.

This example demonstrates that processing a composition comprising tetrahydrocannabinol, a blend of ester type polyethoxylated high HLB surfactants, a blend of low HLB surfactants that include mono- and di-glycerides, and a mixture of terpenes including d-limonene and olive oil unsaponifiables is effective to produce an edible tetrahydrocannabinol

DNLF dispersion with volume average particle diameter less than 50 nm and turbidity below 500 NTU that is capable to induce onset of intoxication from 400 mg within 30 minutes of buccal plus sublingual administration and is capable to avoid intoxication from 200 mg by oral administration.

### Example 7

### Processing of an Edible Mill Base Including Polysorbate 80, % Polysorbate 20, Sodium Caprate, PEG 30 Glyceryl Cocoate, Sorbitan Stearate, Sorbitan Oleate, Soy Lecithin, Medium Chain Triglyceride Oil, Isopropyl Myristate, Ethyl Myristate, d-Limonene, Myrcene, Eugenol, Cannabidiol, Tetrahydrocannabinol and Water Using a Modified Twin Screw Extruder

A mill base was prepared consisting of 38.7% water, 10.7% polysorbate 80, 8.8% medium chain triglyceride oil, 7.0% isopropyl myristate, 6.8% ethyl myristate, 3.6% d-limonene, 3.6% polysorbate 20, 3.5% myrcene, 2.7% sorbitan stearate, 2.7% sorbitan oleate, 2.2% soy lecithin, 1.8% eugenol, 1.8% PEG 30 glyceryl cocoate, 1.7% sodium lauryl sulfate, 1.4% delta 8 tetrahydrocannibinol, 1.3% cannabidiol, 1.0% sodium caprate, 0.2% clove essential oil, 0.2% hydrochloric acid 0.2% sodium benzoate, 0.1% butylated hydroxy toluene, and 0.04% citric acid. The ratio of ether type polyethoxylated high HLB surfactant to ester type polyethoxylated high HLB surfactant was 0:100. The mill base was processed with an extruder inlet temperature equal to 94° C., extruder outlet temperature equal to 24° C. and process ΔT=70° C. at a rate of 530 grams per minute to give a tetrahydrocannabinol lipid nanoparticle dispersion as a transparent soft psuedoplastic gel. The volume average particle diameter (Dv) was 60 nm, the number average particle diameter (Dn) was 49 nm, the polydispersity was 1.2, and the turbidity was 740 NTU. When stored at 40° C., the sample phase separated within 5 days.

This example demonstrates that processing a composition comprising tetrahydrocannabinol, a blend of ester type polyethoxylated high HLB surfactants and anionic high HLB surfactants including polysorbates, sodium lauryl sulfate, sodium caprate, and PEG30 glyceryl cocoate, a blend of ester oils including ethyl myristate and isopropyl myristate, a blend of low HLB surfactants including lecithin and sorbitan esters, and a mixture of terpenes including d-limonene, myrcene and eugenol is effective to produce an edible tetrahydrocannabinol DNLF dispersion with volume average particle diameter less than 100 nm and turbidity below 750 NTU.

### Example 8

### Processing of an Edible Mill Base Including Polysorbate 80, Polysorbate 20, Sodium Stearoyl Lactylate, Hydrogen Stearoyl Lactylate, Sodium Lauryl Sulfate, Soy Lecithin, Glyceryl Monostearate, Stearic Acid, Medium Chain Triglyceride Oil, Methyl Undecylenate, Ethyl Caproate, Ethyl Caprate, Ethyl Myristate, Ethyl Oleate, Isopropyl Myristate, Light Mineral Oil, d-Limonene, Humulene, Cannabidiol, Tetrahydrocannabinol and Water Using a Modified Twin Screw Extruder

A mill base was prepared consisting of 42.7% water 9.1% polysorbate 80, 7.8% isopropyl myristate, 7.1% medium chain triglyceride oil, 6.1% ethyl oleate, 4.7% polysorbate 20, 4.5% d-limonene, 3.9% light mineral oil, 2.1% sodium stearoyl lactylate, 1.7% soy lecithin, 1.7% delta 8 tetrahydrocannibinol, 1.2% cannabidiol, 1.1% sodium lauryl sulfate, 1.0% hydrogen stearoyl lactylate, 1.0% glycerin, 0.9% ethyl myristate, 0.8% glyceryl monostearate, 0.7% humulene, 0.3% methyl undecylenate, 0.3% clove essential oil, 0.2% ethyl caprate, 0.2% ethyl caproate, 0.2% sodium benzoate, 0.1% cinnamon bark essential oil, 0.1% concentrated orange essential oil, 0.1% hydrochloric acid 0.1% stearic acid, 0.1% butylated hydroxy toluene, and 0.03% citric acid. The ratio of ether type polyethoxylated high HLB surfactant to ester type polyethoxylated high HLB surfactant was 0:100. The mill base was processed with an extruder inlet temperature equal to 92° C., extruder outlet temperature equal to 22° C. and process ΔT=70° C. at a rate of 420 grams per minute to give a tetrahydrocannabinol lipid nanoparticle dispersion as a transparent soft psuedoplastic gel. The volume average particle diameter (Dv) was 85 nm, the number average particle diameter (Dn) was 58 nm, and the polydispersity was 1.5. When stored at 40° C., the sample phase separated sometime between 6 and 12 days.

This example demonstrates that processing a composition comprising tetrahydrocannabinol, a blend of ester type polyethoxylated high HLB surfactants and anionic high HLB surfactants including polysorbates, hydrogen stearoyl lactylate, and sodium lauryl sulfate, a blend of low HLB surfactants including lecithin and glyceryl monostearate, a blend of ester oils including methyl undecylenate, ethyl caproate, ethyl caprylate, ethyl myristate, ethyl oleate and isopropyl myristate, and a blend of terpenes including d-limonene and humulene is effective to produce an edible tetrahydrocannabinol DNLF dispersion with volume average particle diameter less than 100 nm.

### Example 9

### Processing of a Mill Base Including Polysorbate 80, Ceteareth-30, Steareth-40, Soy Lecithin, Glyceryl Monostearate, Sorbitan Oleate, Medium Chain Triglyceride Oil, Isopropyl Myristate, Light Mineral Oil, Arnica Flower Oil, Cannabidiol, Tetrahydrocannabinol and Water Using a Modified Twin Screw Extruder

A mill base was prepared consisting of 45.0% water, 14.1% isopropyl myristate, 7.9% light mineral oil, 7.1% polysorbate 80, 5.5% glycerin, 3.3% ceteareth-30, 3.3% medium chain triglyceride oil, 2.6% sorbitan oleate, 2.0% cannabidiol, 1.9% steareth-40, 1.9% arnica flower oil, 1.2% stearic acid, 1.0% glyceryl monostearate, 0.9% delta 8 tetrahydrocannibinol, 0.6% tocopherol, 0.4% Jeecide CAP-4 preservative, 0.3% rosemary-mint fragrance, 0.3% soy lecithin, 0.2% sodium phytate, 0.1% ascorbyl palmitate, 0.1% sodium benzoate, 0.1% clove essential oil, and 0.1% citric acid. The ratio of ether type polyethoxylated high HLB surfactant to ester type polyethoxylated high HLB surfactant was 43:57. The mill base was processed with an extruder inlet temperature equal to 95° C., extruder outlet temperature equal to 18° C. and process ΔT=77° C. at a rate of 340 grams per minute to give a tetrahydrocannabinol lipid nanoparticle dispersion as a transparent soft psuedoplastic gel. The volume average particle diameter (Dv) was 66 nm, the number average particle diameter (Dn) was 38 nm and the polydispersity was 1.7. When a sample of the lipid nanoparticle dispersion was tested for latent lamellar structure, the plot of conductivity vs temperature did not show a negative peak and the sample was birefringent between 70 and 79° C. which indicates that it has latent lamellar structure. When stored at 40° C., the sample phase separated sometime between 14 and 16 days.

This example demonstrates that processing a composition comprising tetrahydrocannabinol, cannabidiol, a blend of ether type polyethoxylated high HLB surfactants and ester type polyethoxylated surfactants including ceteareth-30 and steareth-40 and polysorbate 80, a blend of low HLB surfactants including lecithin, glyceryl monostearate, and sorbitan oleate, a single ester oil, plus medium chain triglyceride oil, arnica flower oil and light mineral oil is effective to produce tetrahydrocannabinol DNLF dispersion suitable for cutaneous application with volume average particle diameter less than 120 nm.

### Example 10

### Processing of a Mill Base Including Polysorbate 80, Polysorbate 20, Sodium Stearoyl Lactylate, Sorbitan Oleate, Soy Lecithin, Stearic Acid, Isopropyl Myristate, Light Mineral Oil, Niacinamide, Tetrahydrocannabinol, Glycerin and Water Using a Modified Twin Screw Extruder

A mill base was prepared consisting of 12.0% polysorbate 80, 2.4% polysorbate 20, 1.0% sodium stearoyl lactylate, 4.0% sorbitan oleate, 1.5% soy lecithin, 2.0% stearic acid, 26.4% isopropyl myristate, 10.8% light mineral oil, 1.1% delta-8 tetrahydrocannabinol, 2.0% niacinamide, 0.1% citric acid, 6.0% glycerin, and 30.9% water. The mill base was processed with an extruder inlet temperature equal to 95° C., extruder outlet temperature equal to 18° C. and process ΔT=77° C. at a rate of 340 grams per minute to give a tetrahydrocannabinol lipid nanoparticle dispersion as a translucent viscous liquid. The volume average particle diameter (Dv) was 55 nm, the number average particle diameter (Dn) was 33 nm and the polydispersity was 1.7 and the turbidity was 150 NTU.

This example demonstrates that processing a composition comprising tetrahydrocannabinol, a blend of low HLB surfactants including lecithin and sorbitan oleate, a blend of high HLB surfactants including polyethoxylated polysorbate 80 and polysorbate 20 and non-polyethoxylated sodium stearoyl lactylate, plus isopropyl myristate and light mineral oil is effective to produce a tetrahydrocannabinol DNLF dispersion suitable for oral administration with volume average particle diameter less than 100 nm and turbidity less than 200 NTU.

### Example 11

### Processing of a Mill Base Including Polysorbate 80, Polysorbate 20, Sodium Stearoyl Lactylate, Sorbitan Oleate, Soy Lecithin, Stearic Acid, Isopropyl Myristate, Light Mineral Oil, Niacinamide, Tetrahydrocannabinol, Glycerin and Water Using a Modified Twin Screw Extruder

A mill base was prepared consisting of 12.0% polysorbate 80, 2.4% polysorbate 20, 1.1% sodium stearoyl lactylate, 3.9% sorbitan oleate, 1.4% soy lecithin, 2.0% stearic acid, 26.4% isopropyl myristate, 11.5% light mineral oil, 0.19% delta-9 tetrahydrocannabinol, 2.0% niacinamide, 0.2% citric acid, 6.0% glycerin, and 30.9% water. The mill base was processed with an extruder inlet temperature equal to 95° C., extruder outlet temperature equal to 18° C. and process delta T=77° C. at a rate of 340 grams per minute to give a tetrahydrocannabinol lipid nanoparticle dispersion as a translucent viscous liquid. The volume average particle diameter (Dv) was 53 nm, the number average particle diameter (Dn) was 32 nm and the polydispersity was 1.7.

This example demonstrates that processing a composition comprising tetrahydrocannabinol, a blend of low HLB surfactants including lecithin and sorbitan oleate, a blend of high HLB surfactants including polyethoxylated polysorbate 80 and polysorbate 20 and non-polyethoxylated sodium stearoyl lactylate, plus isopropyl myristate and light mineral oil is effective to produce a tetrahydrocannabinol DNLF dispersion suitable for oral administration with volume average particle diameter less than 100 nm.

### Example 12

### Processing of a Mill Base Including Polysorbate 80, Glyceryl Monostearate, Sorbitan Oleate, Ascorbyl Palmitate, Tocopherol, Isopropyl Myristate, Light Mineral Oil, Clove Essential Oil, Delta-8 Tetrahydrocannabinol, Delta-9 Tetrahydrocannabinol, Delta-9 Tetrahydrocannabinolic Acid, and Water Using a Modified Twin Screw Extruder

A mill base was prepared consisting of 15.5% polysorbate 80, 1.1% glyceryl monostearate, 2.0% sorbitan oleate, 0.3% ascorbyl palmitate, 0.7% tocopherol, 26.7% isopropyl myristate, 10.9% light mineral oil, 0.6% delta-8 tetrahydrocannabinol, 0.09% delta-9 tetrahydrocannabinol, 0.19% delta-9 tetrahydrocannabinolic acid, 1.0% clove essential oil, 0.2% sodium benzoate, 0.04% citric acid, 1.1% glycerin, and 39.9% water. The mill base was processed with an extruder inlet temperature equal to 95° C., extruder outlet temperature equal to 18° C. and process ΔT=77° C. at a rate of 340 grams per minute to give a tetrahydrocannabinol plus tetrahydrocannabinolic acid lipid nanoparticle dispersion as a translucent viscous liquid. The volume average particle diameter (Dv) was 61 nm, the number average particle diameter (Dn) was 37 nm and the polydispersity was 1.6.

This example demonstrates that processing a composition comprising tetrahydrocannabinol and tetrahydrocannabinolic acid, a blend of low HLB surfactants including glyceryl monostearate and sorbitan oleate, polysorbate 80 plus isopropyl myristate, light mineral oil and clove essential oil is effective to produce a tetrahydrocannabinol plus tetrahydrocannabinolic acid DNLF dispersion suitable for oral administration with volume average particle diameter less than 100 nm.

### Example 13

### Processing of a Mill Base Including Laureth-23, PEG100 Stearate, Dioleyl Phosphatidyl Choline, 3.1% Sorbitan Oleate, Isopropyl Myristate, Limonene, Ibuprofen, Delta-8 Tetrahydrocannabinol, Medium Chain Triglyceride Oil, and Water Using a Modified Twin Screw Extruder

A mill base was prepared consisting of 6.1% laureth-23, 4.6% PEG100 stearate, 0.5% dioleyl phosphatidyl choline (Phospholipon 90G, product of Lipoid), 3.1% sorbitan oleate, 20.1% isopropyl myristate, 5.1% limonene, 5.1% ibuprofen, 1.2% delta-8 tetrahydrocannabinol, 1.0% medium chain triglyceride oil, and 53.2% water. The weight ratio of PEG100 stearate (an ester type polyethoxylated high HLB surfactant) to laureth-23 (an ether type polyethoxylated high HLB surfactant) is 0.75 to 1.0. The mill base was processed with an extruder inlet temperature equal to 95° C., extruder outlet temperature equal to 18° C. and process ΔT=77° C. at a rate of 340 grams per minute to give a tetrahydrocannabinol lipid nanoparticle dispersion as a translucent viscous liquid. The volume average particle diameter (Dv) was 106 nm, the number average particle diameter (Dn) was 69 nm and the polydispersity was 1.5.

When a sample of the lipid nanoparticle dispersion was tested for latent lamellar structure by stirring and heating, the sample became transparent and was birefringent between 56 and 60° C., which indicates that it has latent lamellar structure.

This example demonstrates that processing a composition comprising tetrahydrocannabinol, a blend of polyethoxylated high HLB surfactants including laureth-23 and PEG100 stearate, a blend of low HLB surfactants including dioleyl phosphatidyl choline and sorbitan oleate, isopropyl myristate, limonene, ibuprofen, medium chain triglyceride oil and water is effective to produce a tetrahydrocannabinol DNLF dispersion with latent lamellar structure suitable for topical administration with volume average particle diameter less than 120 nm.

### Example 14

### Processing of a Mill Base Including Laureth-23, PEG100 Stearate, Dioleyl Phosphatidyl Choline, Sorbitan Oleate, Isopropyl Myristate, Limonene, Delta-8 Tetrahydrocannabinol, Medium Chain Triglyceride Oil, and Water Using a Modified Twin Screw Extruder

A mill base was prepared consisting of 6.5% laureth-23, 4.9% PEG100 stearate, 0.5% dioleyl phosphatidyl choline (Phospholipon 90G, product of Lipoid), 3.3% sorbitan oleate, 21.1% isopropyl myristate, 5.4% limonene, 1.2% delta-8 tetrahydrocannabinol, 1.1% medium chain triglyceride oil, and 56.0% water. This is the same composition as DNLF A described in Dense nanolipid fluid dispersions comprising ibuprofen: Single step extrusion process and drug properties. Int J Pharm. 2021 Apr. 1; 598:120289. doi: 10.1016/j.ijpharm.2021.120289 (Morrison et al) except that ibuprofen has been removed and replaced with delta-8 tetrahydrocannabinol. The mill base was processed with an extruder inlet temperature equal to 95° C., extruder outlet temperature equal to 18° C. and process ΔT=77° C. at a rate of 340 grams per minute to give a tetrahydrocannabinol lipid nanoparticle dispersion as an opaque viscous liquid. The volume average particle diameter (Dv) was 3210 nm, the number average particle diameter (Dn) was 125.3 nm and the polydispersity was 26.

When a sample of the dispersion was tested for latent lamellar structure by stirring and heating, it remained opaque and birefringence was not observed from between 50° C. and the boiling point (approximately 100° C.) and there was no negative peak in the plot of conductivity vs temperature, which indicates that the dispersion lacks latent lamellar structure.

What this example demonstrates is that processing a composition comprising tetrahydrocannabinol, a blend of polyethoxylated high HLB surfactants including laureth-23 and PEG100 stearate, a blend of low HLB surfactants including dioleyl phosphatidyl choline and sorbitan oleate, isopropyl myristate, limonene, medium chain triglyceride oil and water is not effective to produce a tetrahydrocannabinol DNLF dispersion with latent lamellar structure when the composition does not contain ibuprofen.

1. A lipid nanoparticle dispersion comprising:
greater than 0.1 percent tetrahydrocannabinol;
one or more high hydrophile-lipophile-balance (HLB) surfactants selected from the group consisting of polysorbate-80, polysorbate-60, polysorbate-20, sodium lauryl sulfate, sodium stearoyl lactylate, PEG-30 glyceryl cocoate, steareth-40, ceteareth-30, laureth-23, PEG100 stearate, and sodium octanoate;
one or more low hydrophile-lipophile-balance (HLB) surfactants selected from the group consisting of lecithin, glyceryl monostearate, sorbitan stearate, sorbitan oleate, stearic acid, and mono- and di-glycerides;
one or more water immiscible oils selected from the group consisting of medium chain triglyceride oil, methyl undecylenate, ethyl caproate, ethyl caprylate, ethyl myristate, ethyl oleate, isopropyl myristate, light mineral oil, d-limonene, myrcene, eugenol, humulene, olive oil unsaponifiables, arnica flower oil, orange essential oil, clove essential oil, and cinnamon bark essential oil; and
water,
wherein said lipid nanoparticle dispersion has a volume average particle size less than about 120 nm and greater than 25 percent lipophilic content.

2. The lipid nanoparticle dispersion of claim 1, wherein the lipid nanoparticle dispersion comprises lipid nanoparticles that are not vesicular nanoparticles.

3. The lipid nanoparticle dispersion of claim 1, wherein the lipid nanoparticle dispersion comprises lipid nanoparticles that do not have a lamellar structure.

4. The lipid nanoparticle dispersion of claim 1, wherein the lipid nanoparticle dispersion has a latent lamellar structure.

5. The lipid nanoparticle dispersion of claim 1, wherein the lipid nanoparticle dispersion further includes one or more cryoprotectants.

6. The lipid nanoparticle dispersion of claim 1, wherein the lipid nanoparticle dispersion is free of ether type polyethoxylated high hydrophile-lipophile-balance (HLB) surfactants.

7. The lipid nanoparticle dispersion of claim 1, wherein the lipid nanoparticle dispersion is free from ibuprofen, S-ibuprofen and lidocaine.

8. The lipid nanoparticle dispersion of claim 1, wherein the turbidity of the lipid nanoparticle dispersion is less than 750 NTU.

9. The lipid nanoparticle dispersion of claim 1, wherein the lipid nanoparticle dispersion is stable to phase separation when stored for 7 days at 40° C.

10. The lipid nanoparticle dispersion of claim 1, wherein tetrahydrocannabinol comprises (6aR,10aR)-6,6,9-trimethyl-3-pentyl-6a,7,8,10a-tetrahydrobenzo[c]chromen-1-ol.

11. The lipid nanoparticle dispersion of claim 1, wherein the concentration of tetrahydrocannabinol is greater than 1%.

12. A process for the preparation of a lipid nanoparticle dispersion comprising conveying a mill base through a twin screw extruder with a process ΔT greater than about 45° C. wherein said mill base comprises:
greater than 0.1 percent tetrahydrocannabinol;
from about 10 to about 16 percent of one or more polyethoxylated high hydrophile-lipophile-balance (HLB) surfactants;
from about 4 to about 8 percent of one or more low hydrophile-lipophile-balance (HLB) surfactants;
from about 25 to about 40 percent of one or more water immiscible oil; and
from about 30 to about 60 percent water,
wherein the lipid nanoparticle dispersion has a volume average particle size less than 120 nm.

13. The process of claim 12 wherein the lipid nanoparticle dispersion has a latent lamellar structure.

14. The process of claim 12 wherein the twin screw extruder barrel comprises an internal cooling channel.

15. The process of claim 12 wherein a fluid with temperature less than 10° C. is pumped through the internal cooling channel of the twin screw extruder barrel during processing of a mill base.

16. The process of claim 12 wherein the mill base is free from ether type polyethoxylated high hydrophile-lipophile-balance (HLB) surfactants.

17. The process of claim 12 wherein all surfactants, water immiscible oils, essential oils, oil soluble vitamins, cryo-protectants, and preservatives present in the mill base with concentrations greater than 0.1% are listed in the US Food and Drug Administration Code of Federal Regulations Title 21 Part 172 Food additives permitted for direct addition to food for human consumption.

18. The process of claim 12 wherein the mill base is free from ibuprofen, S-ibuprofen and lidocaine.

19. The process of claim 12 wherein tetrahydrocannabinol comprises (6aR,10aR)-6,6,9-trimethyl-3-pentyl-6a,7,8,10a-tetrahydrobenzo[c]chromen-1-ol.

20. The process of claim 12 wherein the concentration of tetrahydrocannabinol is greater than 1%.
